(19) United States
(12) Patent Application Publication
(10) Pub. No.: US 2024/0389956 A1
(43) Pub. Date: Nov. 28, 2024

(54) METHOD AND APPARATUS FOR DETERMINING A PCD OUTPUT MODEL IN A COMPUTED TOMOGRAPHY IMAGING SYSTEM

(71) Applicants: CANON MEDICAL SYSTEMS CORPORATION, Tochigi (JP); The Johns Hopkins University, Baltimore, MD (US)

(72) Inventors: Donghyeon LEE, Baltimore, MD (US); Katsuyuki TAGUCHI, Elkridge, MD (US); Xiaohui ZHAN, Vernon Hills, IL (US)

(73) Assignees: CANON MEDICAL SYSTEMS CORPORATION, Tochigi (JP); The Johns Hopkins University, Baltimore, MD (US)

(21) Appl. No.: 18/673,028

(22) Filed: May 23, 2024

Related U.S. Application Data
(60) Provisional application No. 63/504,702, filed on May 26, 2023.

(51) Int. Cl. A61B 6/03; A61B 6/00; A61B 6/42; A61B 6/58

(52) U.S. Cl. CPC A61B 6/032; A61B 6/4241; A61B 6/482; A61B 6/488; A61B 6/582

(57) ABSTRACT

A method for determining a model characterizing an output from a photon-counting detector (PCD) used in a computed tomography (CT) system is provided. The PCD has a plurality of pixels. The method includes constructing a PCD output model that has a plurality of model parameters including a first model parameter set. The first model parameter set is dependent on an incident count rate on the PCD, and dependent on a pixel position in the PCD. The method also includes receiving calibration data acquired by scanning a plurality of combinations of basis materials, under a plurality of incident count rates. The method further includes estimating the plurality of model parameters based on the received calibration data.

### CROSS REFERENCE TO RELATED APPLICATIONS

The present application claims priority to U.S. Provisional Application No. 63/504,702, filed on May 26, 2023. The U.S. Provisional Application is incorporated herein by reference in its entirety.

### FIELD

The present application relates to X-ray computed tomography (CT) imaging systems based on photon-counting detectors (PCDs). Specifically, the application relates to determination of a model for characterizing the output of the PCD used in a CT imaging system.

### DESCRIPTION OF THE RELATED ART

PCD-based CT provides many clinical advantages over the conventional energy-integrating detector CT, thanks to its energy-discriminating capability. However, the responses of a PCD to different X-ray energy levels are not linear, and the X-ray spectrum is distorted due to several factors, such as charge sharing (CS) and pulse pileup (PP). It is critical to characterize the PCD output in order to either compensate or correct for the spectral distortion and fully exploit the merits of the PCDs.

Approaches to characterize PCD data have been data-based or model-based, and data-based approaches are either calibration-based or deep learning-based. Through calibration processes, calibration-based methods estimate parameters for an empirical function such as polynomials for the forward process (i.e., computing the expectation of measured data from a set of variables, e.g., thicknesses of attenuation materials) or the inverse process (returning the set of variables from measured data). Deep learning-based methods use much larger number of parameters than the calibration-based methods and figure out the mapping function from training data. These data-based approaches are simple and can be accurate if scan conditions used for calibration or training match to the scan conditions with object. They are, however, labor-intensive since a large number of calibration or training data is required and the accuracy with untested conditions is not certain.

In contrast, the model-based methods analytically interpret physical detection processes including charge sharing and pulse pileup, and relate upstream variables such as thicknesses of attenuation materials to the expected PCD data. These methods can handle untested conditions, once model parameters are estimated and the behavior of the model is validated. It is very challenging, however, to develop a model that is in good agreement with physical PCD's data. A handful of models have been assessed against physical PCDs, and even these models have shown visual mismatch with the outputs of physical PCDs when both charge sharing and pulse pileup are present. Two challenges in the model-based methods are a remaining model-data mismatch and pixel-to-pixel variations.

Thus, there is a need to alleviate the model-data mismatch issue and to provide a method for creating an enhanced PCD output model.

### SUMMARY

An embodiment of present application is directed to a method for determining a model characterizing an output from a photon-counting detector (PCD) used in a computed tomography (CT) system. The PCD has a plurality of pixels. The method includes constructing a PCD output model that has a plurality of model parameters including a first model parameter set. The first model parameter set is dependent on an incident count rate on the PCD, and dependent on a pixel position in the PCD. The method also includes receiving calibration data acquired by scanning a plurality of combinations of basis materials, under a plurality of incident count rates. The method further includes estimating the plurality of model parameters based on the received calibration data.

Another embodiment of the present application is directed to an apparatus for determining a model characterizing an output from a photon-counting detector (PCD) used in a computed tomography (CT) system. The PCD has a plurality of pixels. The apparatus includes processing circuitry that is configured to construct a PCD output model that has a plurality of model parameters including a first model parameter set, where the first model parameter set is dependent on an incident count rate on the PCD, and dependent on a pixel position in the PCD. The processing circuitry is further configured to receive calibration data acquired by scanning a plurality of combinations of basis materials, under a plurality of incident count rates. The processing circuitry is also configured to estimate the plurality of model parameters based on the received calibration data.

A further embodiment of present application is directed to a non-transitory computer-readable medium storing a program that, when executed by processing circuitry, causes the processing circuitry to execute a method for determining a model characterizing an output from a photon-counting detector (PCD) used in a computed tomography (CT) system. The PCD has a plurality of pixels. The method includes constructing a PCD output model that has a plurality of model parameters including a first model parameter set. The first model parameter set is dependent on an incident count rate on the PCD, and dependent on a pixel position in the PCD. The method also includes receiving calibration data acquired by scanning a plurality of combinations of basis materials, under a plurality of incident count rates. The method further includes estimating the plurality of model parameters based on the received calibration data.

### BRIEF DESCRIPTION OF THE DRAWINGS

The application will be better understood in light of the description, which is given in a non-limiting manner, accompanied by the attached drawings in which:

FIG. 1 shows a flow chart of a PCD model developing process 100 according to one embodiment of the present application;

FIG. 2 shows a flow chart of a calibration process 200 of the PCD output model, according to one embodiment of the present application;

FIG. 3 shows the structure of a PCD detecting X-rays that are emitted from the X-ray tube and pass through an imaging object;

FIG. 4A shows a scatter plot of 30 data points used for model parameter estimation (26 data points) and validation (4 data points) plotted over water thicknesses, aluminum thicknesses, and tube current values;

FIG. 4B shows an initial 120 kVp spectrum and the most attenuated spectrum;

FIG. 5A shows the measured count-rates and count-rates from the modified cascaded PCD model with the model parameters obtained in Step 1;

FIG. 5B shows the fitting results for the global threshold functions, E_{glob}(α, c_{b}) obtained in Step 3;

FIG. 6 shows measured PCD data and the output of three PCD models at the lowest count-rate condition among the test data with 10 mA, 16 cm of water, and 0.5 cm of aluminum;

FIG. 7 shows measured PCD data and the output of the three PCD models at the highest count-rate condition with blank scan at 100 mA;

FIGS. 8A-8D show mean absolute percentage errors (MAPEs, left axes) for the three models calculated for each bin, each of the four-test data, and coefficients of variation (CVs, broken lines, right axes) for the measured data;

FIG. 9 shows the MAPEs of the three PCD models, which are calculated with respect to the incident count-rate of all of the test data; and

FIG. 10 shows an example of a photon-counting CT scanner system that can incorporate the techniques disclosed herein.

### DETAILED DESCRIPTION

Embodiments or examples for implementing various aspects of the present application may be as set forth in the following sections. Specific examples of components and arrangements are described below to simplify the present application. These are, of course, merely examples and are not intended to be limiting.

The order of discussion of the different steps as described herein is presented for the sake of clarity. In general, these steps can be performed in any suitable order. Additionally, although each of the different features, techniques, configurations, etc. herein may be discussed in different places of this application, it is intended that each of the concepts can be executed independently of each other or in combination with each other. Accordingly, the present application can be embodied and viewed in many different ways.

Furthermore, as used herein, the words “a,” “an,” and the like generally carry a meaning of “one or more,” unless stated otherwise.

In order to overcome the aforementioned challenges, this application introduces a method and apparatus for the development of a highly accurate PCD output model. By incorporating count-rate-dependent threshold energy functions, the method and apparatus effectively address the global model-data mismatch of the cascaded model, as well as the pixel-to-pixel variations. The resulting PCD output model provides precise estimations of physical PCD outputs.

The PCD output model demonstrates exceptional agreement with the measured PCD data, exhibiting mean absolute percentage errors (MAPEs) of less than 5% for all test data, and even lower than 2% for the majority of datasets. On average, the MAPEs across all bins range from 1.1% to 2.4%. It is anticipated that this model will significantly contribute to applications where pixel-by-pixel accuracy is important.

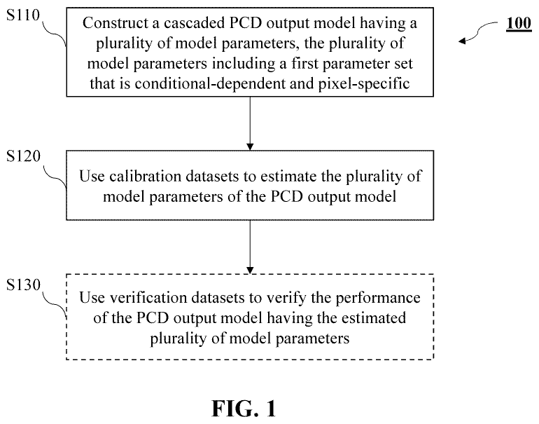

FIG. 1 shows a flow chart of a PCD model developing process 100 according to one embodiment of the present application. In step S110, a cascaded PCD output model is constructed. The model can comprise a plurality of model parameters, including a first parameter set that is conditional-dependent and pixel-specific. In step S120, the plurality of model parameters of the PCT output model can be estimated through a calibration process, based on calibration datasets. Optionally, in step S130, the performance of the PCT output model having the estimated parameters can be verified based on verification datasets. The developed model can be used to simulate the output of the PCD, enabling accurate representation of the PCD's behavior. Additionally, the developed model can be used to correct or compensate for spectral distortions in the measured PCD output data, ensuring reliable and precise measurement results.

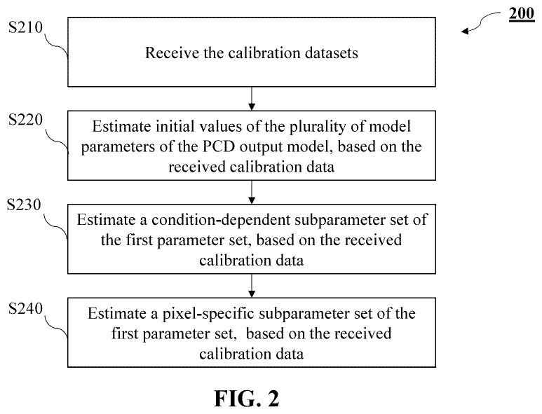

FIG. 2 shows a flow chart of a calibration process 200 of the PCD output model, according to one embodiment of the present application. The process begins with step S210, where the calibration datasets are obtained. These datasets, along with the verification datasets used in the optional step S130 of FIG. 1, can be acquired from tests conducted across various combinations of basis materials and multiple X-ray tube currents. In step S210, initial values of the plurality of model parameters can be estimated based on the received calibration data. In step S230, a condition-dependent subparameter set of the first parameter set can be estimated; and in step S240, a pixel-specific subparameter set of the first parameter set can be estimated.

The details of the processes shown in FIGS. 1 and 2 can be found in the following sections.

### I. Cascaded PCD Output Model

The cascaded PCD output model describes four processes that change the X-ray spectrum sequentially and outputs the final spectrum: (1) attenuation by Beer's law, (2) the charge sharing effect, which can be estimated from a Monte Carlo simulation, (3) the pulse pileup effect, and (4) the energy binning process.

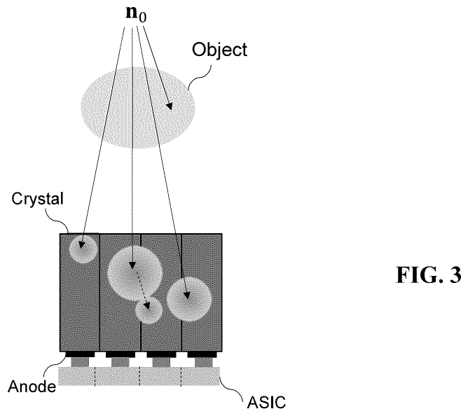

FIG. 3 shows the structure of a PCD detecting X-rays that are emitted from the X-ray tube and pass through an imaging object. Let n be an X-ray spectrum vector and its elements, n_{E}, represent the number of photons within a hypothetical 1-keV-width energy window at energy E, E∈{1, 2, . . . , Eₘₐₓ} keV. The output of the cascaded PCD model, y, can be computed from the spectrum vector emitted from the X-ray tube, n₀, as follows:

$$y = {Ψ}_{B} ( {Ψ}_{PP} ( {Ψ}_{CS} ( {Ψ}_{A} ( {n}_{0} , v ) ) , τ ) , {E}_{thr} ) , \qquad ( 1 )$$

where v denotes a set of basis material thickness vectors (mm), τ is detector deadtime(s), and Eₜₕᵣ is a threshold energy vector (keV), Eₜₕᵣ=(E_{thr,1}, . . . , E_{thr, B})^{T}, B is the number of energy bins for the PCD, and superscript T denotes a transpose. The initial spectrum vector, n₀, can be computed as

$$n 0 = \frac{{k}_{0} \times dA \times I}{{R}^{2}} \times {S}_{0} ,$$

where S₀ refers to the probability mass function of the X-ray spectrum, k₀ is an X-ray photon output rate (counts/s/mA/mm² at 1 m from the X-ray focal spot), dA is a detector pixel area (mm²), I is a tube current (mA), and R is a source-to-detector distance (m). Among all the parameters, k₀ and t are the only parameters that need to be estimated through calibration in the original model. The four operators, Ψ_{A}, Ψ_{CS}, Ψ_{PP}, and Ψ_{B} are explained in the following.

### A. The Attenuation Operator Ψ_{A}

The operator Ψ_{A} computes the spectrum vector after attenuation, n_{A}, using Beer's law as:

$$\\ \begin{matrix}{n}_{A} = {Ψ}_{A} ( {n}_{0} , v ) = M ( v ) n , & ( 2 )\end{matrix}$$

where M(v) is an attenuation matrix, M(v)=diag(exp(−μ₁·v), . . . , exp(−μEₘₐₓ·v)), diag is the diagonal operator, and μ_{E}V=μ_{1,E}ν₁+μ_{2,E}ν₂, where HE is a linear attenuation coefficient vector (mm⁻¹), which elements are linear attenuation coefficients of two basis materials at X-ray energy E.

### B. The Charge Sharing Operator Ψ_{CS}

The operator Ψ_{CS} outputs the spectrum vector after charge sharing, n_{CS}:

$$\\ \begin{matrix}{n}_{CS} = {Ψ}_{CS} ( {n}_{A} ) = {Hn}_{A} & ( 3 )\end{matrix}$$

where H is a two-dimensional matrix that models the effect of CS, where each column of H represents the expected X-ray spectrum given a single count input at a specific X-ray energy in PCDs. H can be obtained by a Monte Carlo simulation.

The Monte Carlo simulation can include (i) photoelectric effect interaction, (ii) Compton scattering, and (iii) penetration with no interaction. Fluorescence X-ray emission after interaction can also be considered. H can contain the effects of both spill-in crosstalk and spill-out crosstalk from neighboring PCDs for the primary interaction, the secondary interaction and so on. It should be noted that the sum of each column of H can be higher than 1 due to the spill-in crosstalk.

### C. The Pulse Pileup Operator Ψ_{PP}

The operator Ψ_{PP} returns the spectrum vector after pulse pileup, n_{PP} for an input spectrum vector n_{CS} and detector deadtime τ:

$$\\ \begin{matrix}{n}_{PP} = {Ψ}_{PP} ( {n}_{CS} , τ ) , & ( 4 )\end{matrix}$$

It can be expressed by a product of three probabilities as follows:

$$\\ \begin{matrix}{n}_{PP , E} = {Ψ}_{PP , E} ( {n}_{CS} , τ ) = a \times Δ t \times Pr ( rec \vert a τ ) \times {\sum }_{m = 0}^{∞} Pr ( m \vert rec ) Pr ( E \vert m ) , & ( 5 )\end{matrix}$$

where the subscript E refers to the E^{th} element for a spectrum vector or a function output. A non-paralyzable detection model can be used, and Pr(rec|ατ) is the probability of events being recorded given by

$$\\ \begin{matrix}Pr ( rec \vert a τ ) = \frac{1}{1 + a τ} , & ( 6 )\end{matrix}$$

where α is the incident count-rate, a=Σ_{E}_{_{thr,n}}^{E}^{ₘₐₓ}Ψ_{CS}(Ψ_{A}(n₀, ν)). E_{thr,n} is a threshold energy for noise cut-off, e.g., 10 keV.

Pr(m|rec) is the probability of the m^{th}-order pulse pileup event, given the events-of-interest being recorded and is given by

$$\\ \begin{matrix}Pr ( m \vert rec ) = \frac{{( a τ )}^{m} exp ( - a τ )}{m !} , & ( 7 )\end{matrix}$$

Lastly, Pr(E|m) is the probability of the recorded energy E, given the m^{th}-order pulse pileup event. The probability is sequentially calculated from the lowest order pulse pileup (m=0) to higher ones. The recorded energy is assumed to be the maximum pulse height of the summed triangular pulses, where each triangular pulse has the width of weighted dead time and the height of the X-ray energy.

### D. The Energy-Binning Operator Ψ_{B}

The operator Ψ_{B} computes the PCD output by integrating the spectral vector with the given threshold energy vector as below:

$$\\ \begin{matrix}y = {Ψ}_{B} ( {n}_{PP} , {E}_{thr} ) = {( {y}_{1} , … , {y}_{B} )}^{T} = {( {\sum }_{{E}_{thr , 1}}^{{E}_{thr , 2}} {n}_{PP , E} , … , {\sum }_{{E}_{thr , B}}^{{E}_{max}} {n}_{PP , E} )}^{T} . & ( 8 )\end{matrix}$$

### II. Global and Local Model Parameters

Through a test of the cascaded PCD output model at various count-rates, the following observations regarding the model-data mismatch can be made: (1) The global mismatch for energy bin data is smaller when count-rates are smaller and increases with increasing count-rates; (2) The total counts are in a good agreement over the entire count-rate range investigated; (3) There exists significant pixel-to-pixel variation, even at low count-rates; and it is somewhat consistent regardless of count-rates, although there exists minor count-rate-dependent changes.

Observations (1)-(2) suggest the presence of count-rate-dependent effect the above cascaded PCD model has not taken into account. These observations also imply that the count-rate-dependent effect can be attributed to the spectral distortion effect above the lowest threshold energy, not the total counts. Observation (3) indicates that the local mismatch is mostly caused by count-rate-independent effects.

Given the above observations, it can be concluded that the causes of the two sorts of mismatches must be different from each other. The global mismatch may be caused by either a count-rate-dependent baseline shift of the pulse train (i.e., the remaining effect even with baseline restoration employed), simply a mismatch between the PP model and the actual effect of PP with PCDs, or other unknown non-linear spectral effect. The local mismatch may be attributed to a mismatch between H used in the CS model and the actual effect of CS or other unknown factors.

Therefore, it is desirable to address the global and local mismatches separately, since not only their causes but also possible changes in the future can be different between the two mismatches.

There are five parameter sets in the above cascaded PCD output model: S₀, k₀, H, T, and Eₜₕᵣ. According to implementations of this application, Eₜₕᵣ can be optimized for conditions. Note that this is not restrictive, as H and/or t can be optimized for conditions in other implementations.

### A. Global Threshold Energies for Global Mismatch

As mentioned above, the threshold energy, Eₜₕᵣ, can be chosen to be count-rate-dependent variables, deviating from the values calibrated at very low count-rates. The global changes of threshold energies can be modeled using exponential functions, because the optimal threshold energies that minimized the global model-data mismatch for various count-rates decreases monotonically with increasing the incident count-rates.

The count-rate-dependent threshold energy for energy bin b can be modeled by:

$$\\ \begin{matrix}{E}_{thr , b} = {E}_{glob} ( a , {c}_{b} ) , & ( 9 )\end{matrix}$$

$$\\ \begin{matrix}{E}_{glob} ( a , {c}_{b} ) = {c}_{b , 0} {e}^{{}_{} - {c}_{b , 1} a} + {c}_{b , 2} . & ( 10 )\end{matrix}$$

Let C=(c₁, . . . , c_{B}) be a set of parameters for all bins; the same C was used for all pixels.

### B. Local Threshold Energies for Local Mismatch

Second-order polynomials, E_{local}, can be added to the global threshold energies in order to model threshold energies for each pixel i; thus, the threshold energy for bin b and pixel i, Ethr,b,i, can be computed by:

$$\\ \begin{matrix}{E}_{thr , b , i} = {E}_{glob} ( a , {c}_{b} ) + {E}_{local} ( a , {d}_{b , i} ) , & ( 11 )\end{matrix}$$

$$\\ \begin{matrix}{E}_{local} ( a , {d}_{i , b} ) = {d}_{b , i , 0} + {d}_{b , i , 1} a + {d}_{b , i , 2} {a}^{{}_{} 2} . & ( 12 )\end{matrix}$$

Let Dᵢ=(d_{1,i}, . . . , d_{B,i}) be a set of parameters for all of bins for pixel i.

### III. Exemplary Procedure for Model Parameter Estimation

As described above, the modified cascaded PCD output model has seven parameter sets: S₀, k₀, H, T, Eₜₕᵣ, C and Dᵢ. S₀ and H can be obtained from the specifications of the CT imaging system. The other model parameters can be estimated by the calibration process.

The PCD data used in the calibration process can be acquired with different thicknesses of basis materials at multiple X-ray tube currents. For example, the PCD can be placed at about 1 m away from the X-ray tube, the tube voltage can be set at 120 kV, and the tube currents can be set at 10, 25, 50, 100, 200, and 400 mA. For each dataset, 1,200 projections obtained for a second can be averaged to decrease the effect of noise; thus, Δt in Eq. (5) is 1/1200 s.

In total of 30 datasets can be acquired with a combination of air, water, and aluminum slabs with different thicknesses, including five blank scan datasets at (25, 50, 100, 200, and 400 mA), three datasets at 50 mA with water of (1, 6, and 16 cm), and 11 different thickness combinations each at either 10 mA or 50 mA. The 11 combinations for water thickness and aluminum thickness (cm, cm) can be (1, 0), (2, 0), (4, 0), (6, 0), (9, 0), (9, 0.5), (12, 0), (12, 0.5), (16, 0), (16, 0.5), and (16, 1). The effective X-ray energy range can be [57.6, 74.4] keV.

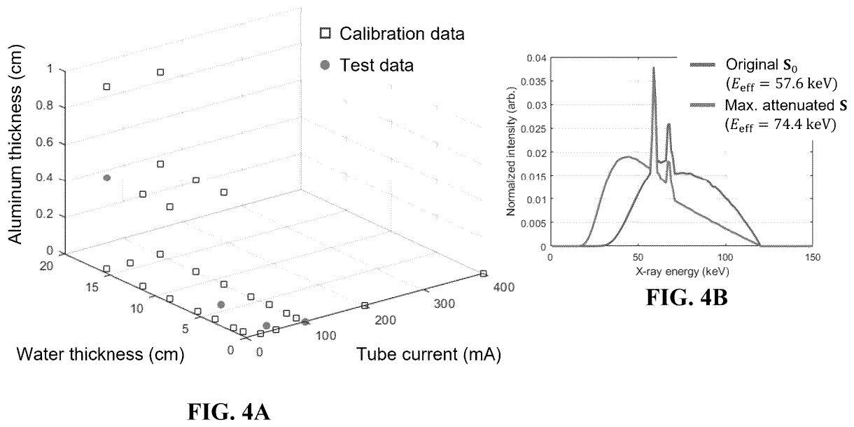

FIG. 4A shows a scatter plot of 30 data points used for model parameter estimation (26 data points) and validation (4 data points) plotted over water thicknesses, aluminum thicknesses, and tube current values. In FIG. 4B, the initial 120 kVp spectrum and the most attenuated spectrum among the data are presented with their effective X-ray energies. These 30 datasets can provide sufficiently large range of count-rates and attenuated spectra.

It would be computationally expensive if one tries to estimate the global and local model parameters (i.e., threshold energies) by minimizing the error between the model output and measured data y_{b,i}^{m}(α), because it would require calculating the model outputs for all pixels for all data in every optimizing step. To enhance the computational efficiency of the calibration process, a surrogate strategy can be employed.

Firstly, optimal threshold energies for the PCD output model that maximize the agreement to measured energy bin output (i.e., photon counts) can be obtained for each bin, each pixel, and each dataset. Then, a count-rate-dependent function for threshold energies can be used, and the parameters of the function can be estimated by maximizing the agreement in threshold energies.

In one embodiment of the application, the calibration process can include four steps, which will be described below. Note that the term “trimmed pixels” in this application refers to pixels within 3 standard deviations from the mean counts across all datasets.

Step 1 (k₀, τ, and E_{thr,1}⁽⁰⁾): Three model parameters, k₀, τ, and the lowest threshold energy, E_{thr,1}⁽⁰⁾, can be estimated by minimizing a squared sum difference in total counts between the PCD output model y_{b} and the mean of trimmed pixels y̅_{b}^{m}:

$$\\ \begin{matrix}{k}_{0}^{{}_{} *} , {τ}^{{}_{} *} , {E}_{thr , 1}^{{}_{} ( 0 ) *} = {arg min}_{{k}_{0} , τ , {E}_{thr , 1}^{{}_{} ( 0 )}} {\sum }_{a} {( {\sum }_{b = 1}^{B} {y}_{b} ( a , {E}_{thr}^{{}_{} ( 0 )} ) - {\sum }_{b = 1}^{B} \overline{y} {}_{b}^{{}_{} m} ( a ) )}^{2} . & ( 13 )\end{matrix}$$

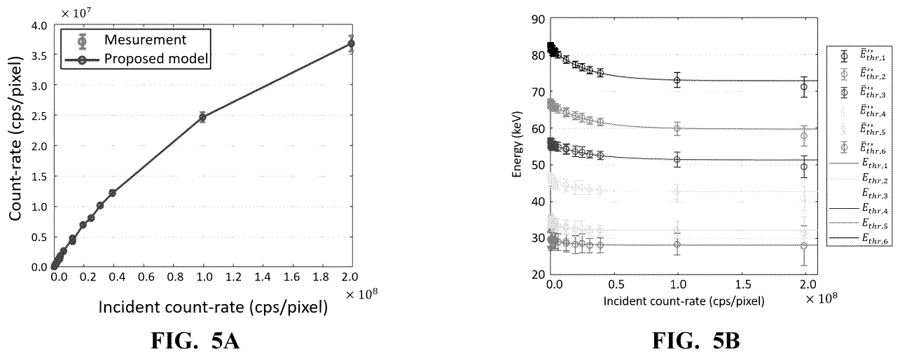

FIG. 5A shows the measured count-rates and count-rates from the modified cascaded PCD model with the model parameters obtained in Step 1. Each of the measured count-rates and its corresponding error bar are the averaged sum of all bin counts over T and the standard deviation at each scan condition, respectively.

Step 2 (E′_{thr,b,i}): Let E′_{thr,b,i}=(E′_{thr,1,i}, . . . , E′_{thr,B,i}) be a vector of threshold energies for B energy bins of pixel i. The optimal E′_{thr,b,i} can be obtained by:

$$\\ \begin{matrix}{E}_{thr , i}^{{}_{} ′ *} ( a ) = {arg min}_{{E}_{thr , i}^{{}_{} ′} ( a )} {‖ {y}_{i} ( a , {E}_{thr , i}^{{}_{} ′} ( a ) ) - {y}_{i}^{{}_{} m} ( a ) ‖}_{2}^{2} , & ( 14 )\end{matrix}$$

where E′*_{thr,i}(α) is jointly optimized and unique.

Step 3 (global C): A set of parameters c_{b} in Eq. (10) can be determined by minimizing the sum of squared difference for each bin b between the global threshold energies E_{glob}(α, c_{b}) and the mean of the optimal threshold energies over pixels

$$\\ \overline{E} {}_{thr , b}^{{}_{} ′ *} ( a ) = \frac{{\sum }_{i} {E}_{thr , b , i}^{{}_{} ′ *} ( a )}{{\sum }_{i} 1} ,$$

$$\\ \begin{matrix}{C}^{{}_{} *} = {arg min}_{C} {\sum }_{b = 1}^{B} {\sum }_{a} {( {E}_{glob} ( a , {c}_{b} ) - \overline{E} {}_{thr , b}^{{}_{} ′ *} ( a ) )}^{2} . & ( 15 )\end{matrix}$$

FIG. 5B shows the fitting results for the global threshold functions, E_{glob}(α, c_{b}) obtained in Step 3. Each of the marks and its corresponding error bar represent the mean of the b^{th} bin optimal energy threshold Ē′*_{b} and the standard deviations of Ē′*_{thr,b,i} over T, respectively.

Step 4 (local Dᵢ): The pixel-specific deviation can be computed from the mean of the trimmed pixels, ΔE_{thr,b,i}=Ē′*_{thr,b}(α)−E′*_{thr,b,i}(α). Then, the parameters Dᵢ in Eq. (13) can be optimized for each detector pixel i by

$$\\ \begin{matrix}{D}_{i}^{{}_{} *} = {arg min}_{{D}_{i}} {\sum }_{b = 1}^{B} {\sum }_{a} {( {E}_{local} ( a , {d}_{b , i} ) - Δ {E}_{thr , b , i} ( a ) )}^{2} . & ( 16 )\end{matrix}$$

The performance of the following three models can be compared: (a) the PCD output model with the default threshold energies and estimated other model parameters, called “default model” in the below; (b) the PCD model with threshold energies optimized globally using Eq. (10), called “global model”; and (c) the PCD model with threshold energies optimized both globally and locally using Eq. (12), called “local model.”

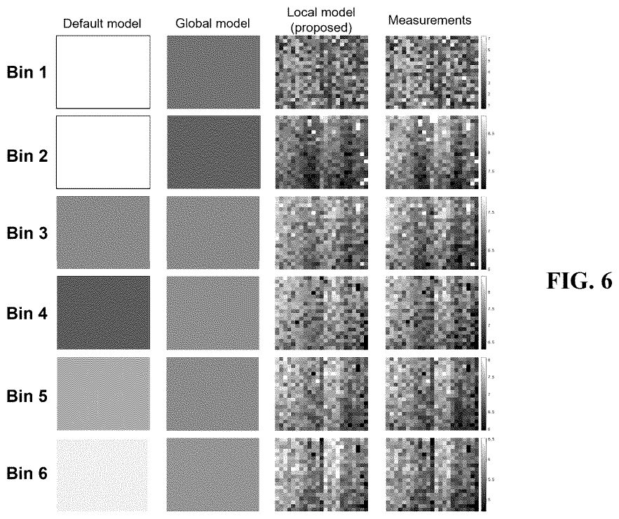

FIG. 6 shows measured PCD data and the output of these three PCD models at the lowest count-rate condition among the test data with 10 mA, 16 cm of water, and 0.5 cm of aluminum. The display window for each bin can be [μ_{b}−2.5σ_{b}, μ_{b}+2.5σ_{b}], where μ_{b} and σ_{b} are the mean and standard deviation of the corresponding bin measurement over pixels, respectively. The same display window width/level centering at the mean of measured PCD data for each bin can be used for all of the data (i.e., medium gray color indicates the mean).

The measured PCD data (rightmost column) presents significant pixel-to-pixel variation with a pattern somewhat consistent among six bins. The default model's values (leftmost column) deviates from the mean of the measured data, with bins 1-2 and 6 being larger (brighter gray colors), and bin 4 being smaller (darker color). Bins 3 and 5 appear good.

The global model outputs (second left) are in good agreement with the mean of PCD measurements, although they do not model the outputs of the individual pixels, as seen in uniform colors.

In contrast, the local model presents striking resemblance to the measured PCD data for each pixel, each bin, even though significant pixel-to-pixel variations exist in the measurement.

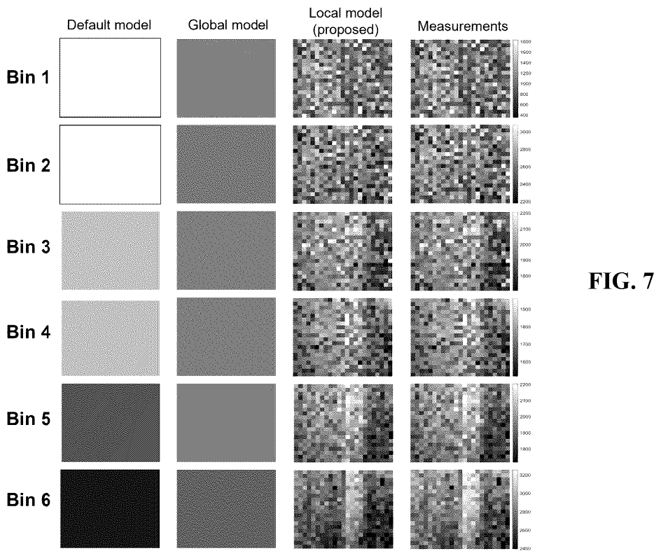

FIG. 7 shows measured PCD data and the output of the three PCD models at the highest count-rate condition with blank scan at 100 mA, where the probability of events being recorded is 0.51. The display window for each bin is [μ_{b}−2.5σ_{b}, μ_{b}+2.5σ_{b}], where μ_{b} and σ_{b} are the mean and standard deviation of the corresponding bin measurement over pixels, respectively.

As can be seen from FIG. 7, the measured PCD data presents significant pixel-to-pixel variations with a pattern less consistent among six bins than FIG. 6. The default model has higher values for bins 1-2 and lower values for bin 6. The global model is in excellent agreement with the means of the measured data for all of the six bins. The local model has excellent agreement with the measured PCD data for all pixels, all of bins. Pixel-to-pixel variations can be correctly captured by the local model.

The goodness of the model-data agreement can be assessed with respect to PCD output (counts) using MAPE, which can be calculated either for each bin independently or for all the bins by:

$$\\ \begin{matrix}{MAPE}_{b} = \frac{100 \%}{n ( T )} {\sum }_{i ∈ T} \vert \frac{{y}_{b , i}^{{}_{} m} - {y}_{b , i}}{{y}_{b , i}^{{}_{} m}} \vert and & ( 17 )\end{matrix}$$

$$\\ \begin{matrix}MAPE = \frac{100 \%}{Bn ( T )} {{\sum }_{b = 1}}^{B} {\sum }_{i ∈ T} \vert \frac{{y}_{b , i}^{{}_{} m} - {y}_{b , i}}{{y}_{b , i}^{{}_{} m}} \vert , & ( 18 )\end{matrix}$$

respectively, where n(T) is the number of the trimmed pixels. The standard error of MAPE can be computed by using bootstrapping as follows. Bootstrap resampling can be performed 1,000 times; the above process can be performed for each resampled data, producing 1,000 MAPE values. The standard error of MAPE represents the standard deviation over 1000 MAPE values. The coefficients of variation (CV) of counts also can be calculated for each bin measurement, as CV allows for relative comparison of bin measurements with respect to their pixel-to-pixel variations.

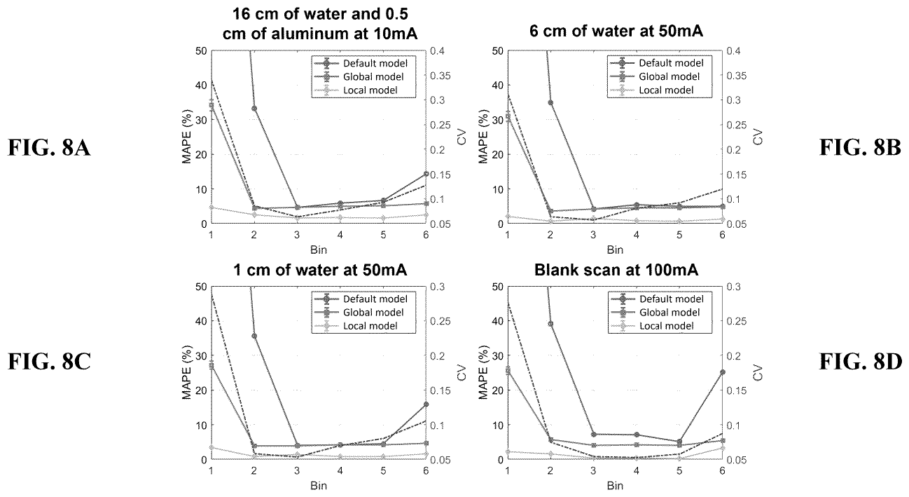

FIGS. 8A-8D show MAPEs (left axes) for the three models calculated for each bin, each of the four-test data, and CVs (broken lines, right axes) for the measured data.

For all the test data, the default model shows good agreement (i.e., MAPEs of 5-10%) for bins 3-5 (i.e., 45-80 keV range), whereas model-data mismatch was significant (MAPE>30%) for bins 1-2 (20-45 keV). The MAPEs of the global model for bins 1-2 and 6 are significantly lower than the default model; however, they are still large in the range of 25-35% for bin 1, which can be attributed to substantial pixel-to-pixel variation indicated by the CVs computed for the measure data. It appears that MAPEs of the global model and CVs of measured data are highly correlated. The MAPEs of the local model are less than 5% for all of the test data and even less than 2% for most datasets.

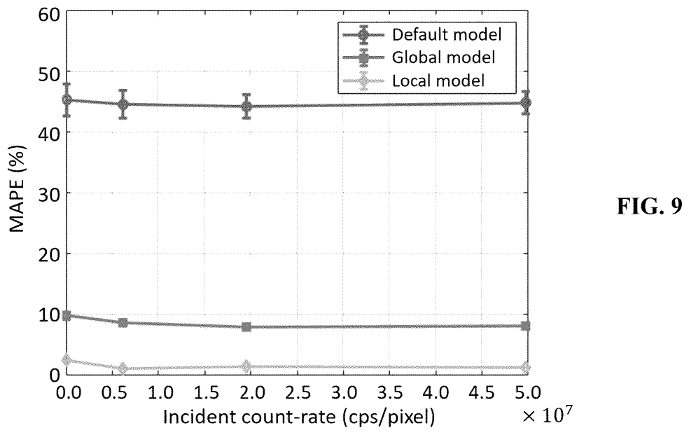

FIG. 9 shows the MAPEs of the three PCD models, which are calculated with respect to the incident count-rate of all of the test data and plotted against a. The three models have consistent error percentages almost independent of the incident count-rates. The MAPEs are 44.1-45.2% for the default model, 8.0-9.8% for the global model, and 1.1-2.4% for the local model.

From the comparison shown in FIGS. 6-9, it is clear that the global model is effective in addressing the global model-data mismatch, while the local model is effective in resolving pixel-to-pixel variation. For the local model, the MAPEs for each bin are lower than 5% for all test data and the MAPEs averaged over all the bins are 1.1-2.4%. Thus, the developed PCD output model can address both the global model-data mismatch and the local model-data mismatch, i.e., pixel-to-pixel variations. Additionally, the MAPEs for the three models are consistent for different incident count-rates and x-ray beam filtrations. It shows that the performance of the developed PCD model is robust against changes of X-ray spectra as well as changes of incident count-rates because the four-test data were acquired with different material thicknesses.

One skilled in the art can envision various potential applications of the developed PCD method in CT imaging. One example of these applications can be a simulator for estimating the output data of the PCD under a specified condition in CT imaging. The user of the simulator can input various imaging parameters, including but not limited to, X-ray tube settings, PCT settings, imaging material thicknesses, etc. to obtain a simulated PCD output. The remarkable interpretation for each fundamental detection mechanism provided by the developed PCD model will be of great help in creating such a PCD simulator.

Another exemplary application is the development of correction or compensation methods to mitigate the effects of charge sharing and pulse pileup in CT imaging. The developed PCT model can be incorporated into material decomposition or reconstruction algorithms of the CT imaging system, for example.

By integrating the method of this application with the data processing pipeline of the CT system, the developed PCD output model can aid in generating output data without spectral distortions. Thus, the impacts caused by charge sharing and pulse pileup in the PCD output data (or reconstructed images) can be corrected or compensated for. This approach will lead to improved image quality, enhanced contrast resolution, and reduced noise. The model's efficient parameter estimation and accuracy also provide practical advantages to algorithms based on it.

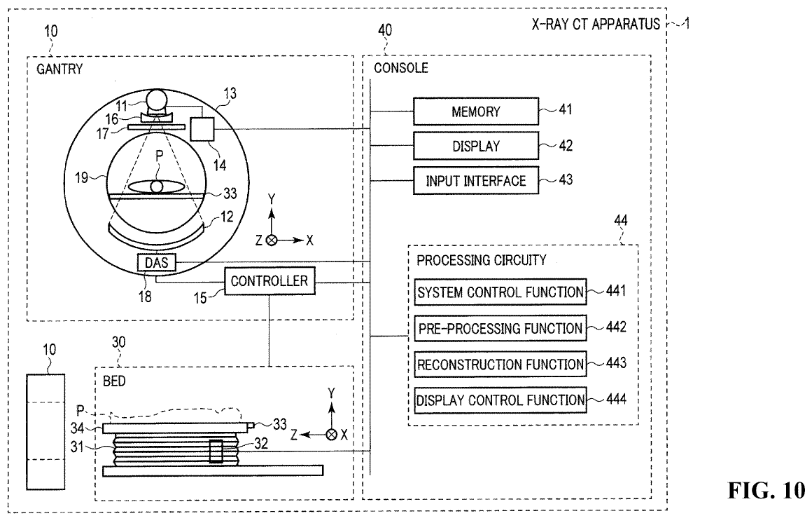

The model determination, correction and/or compensation algorithms can be implemented in a photon-counting CT scanning system as described below with reference to FIG. 10. The X-ray CT apparatus 1 shown in FIG. 10 includes a gantry 10, a bed 30, and a console 40 that implements the processing of the medical imaging processing apparatus. For the sake of explanation, FIG. 10 shows multiple gantries 10.

In the present embodiment, the rotation axis of a rotation frame 13 in the non-tilted state, or the longitudinal direction of a table top 33 of the bed 30, is defined as a “Z-axis direction;” the axial direction orthogonal to the Z-axis direction and horizontal to the floor is defined as an “X-axis direction;” and the axial direction orthogonal to the Z-axis direction and vertical to the floor is defined as a “Y-axis direction.”

For example, the gantry 10 and the bed 30 are installed in a CT examination room, and the console 40 is installed in a control room adjacent to the CT examination room. The console 40 is not necessarily installed in the control room. For example, the console 40 can be installed together with the gantry 10 and the bed 30 in the same room. In any case, the gantry 10, the bed 30, and the console 40 are communicably connected to one another by wire or radio.

The gantry 10 is a scanner with a configuration for performing X-ray CT imaging on a subject (or an imaging object) P. The gantry 10 includes an X-ray tube 11, an X-ray detector 12, a rotation frame 13, an X-ray high voltage device 14, a controller 15, a wedge filter 16, a collimator 17, and a data acquisition system (DAS) 18.

The X-ray tube 11 is a vacuum tube that generates X-rays by emitting thermal electrons from the cathode (filament) to the anode (target) in response to application of a high voltage and supply of a filament current from the X-ray high voltage device 14. Specifically, X-rays are generated by the thermal electrons colliding with the target. Examples of the X-ray tube 11 include a rotating anode type X-ray tube that generates X-rays by emitting thermal electrons to the rotating anode. The X-rays generated in the X-ray tube 11 are, for example, formed into a cone-beam shape by the collimator 17, and applied to the subject P.

The X-ray detector 12 detects X-rays that have been emitted by the X-ray tube 11 and have passed through the subject P, and outputs an electrical signal corresponding to the X-ray dose to the DAS 18. The X-ray detector 12 includes a plurality of X-ray detection element lines, each including a plurality of X-ray detection elements aligned in a channel direction (the X-axis direction, or the column direction) along an arc having a center at the focus of the X-ray tube 11, for example. The X-ray detector 12 has an array structure in which a plurality of X-ray detection element lines, each including a plurality of X-ray detection elements aligned in the channel direction, are aligned in a slice direction (the Z-axis direction, or the row direction).

Specifically, the X-ray detector 12 can be, for example, a direct conversion type detector including a semiconductor element that converts incident X-rays into an electrical signal. The X-ray detector 12 is an example of the PCD according to the present embodiment, and will also be referred to as a “PCD 12.”

The rotation frame 13 supports an X-ray generator and the X-ray detector 12 rotatably around a rotation axis. Specifically, the rotation frame 13 is an annular frame that supports the X-ray tube 11 and the X-ray detector 12 in such a manner that the X-ray tube 11 faces the X-ray detector 12, and rotates the X-ray tube 11 and the X-ray detector 12 under the control of a controller 15 to be described later. The rotation frame 13 is rotatably supported by a stationary frame (not shown) made of a metal such as aluminum. Specifically, the rotation frame 13 is connected to an edge portion of the stationary frame via a bearing. The rotation frame 13 rotates around the rotation axis Z at a predetermined angular velocity while receiving power from a driver of the controller 15.

In addition to the X-ray tube 11 and the X-ray detector 12, the rotation frame 13 includes and supports the X-ray high voltage device 14 and the DAS 18. Such a rotation frame 13 is housed in an approximately-cylindrical case with a bore 19 constituting an imaging space. The bore approximately corresponds to the FOV. The central axis of the bore corresponds to the rotation axis Z of the rotation frame 13. Detection data generated by the DAS 18 is transmitted, for example, from a transmitter (not shown) to a receiver (not shown) arranged on a non-rotating portion (such as the stationary frame, illustration omitted in FIG. 10) of the gantry, and then transferred to the console 40.

The X-ray high voltage device 14 includes: a high voltage generator including electrical circuitry such as a transformer, a rectifier, etc. and having the function of generating a high voltage to be applied to the X-ray tube 11 and a filament current to be supplied to the X-ray tube 11; and an X-ray controller configured to control an output voltage in accordance with the X-rays emitted by the X-ray tube 11. The high voltage generator can be of a transformer type, or an inverter type. The X-ray high voltage device 14 may be provided in the rotation frame 13 to be described later, or in the stationary frame (not shown) of the gantry 10.

The controller 15 includes processing circuitry including a central processing unit (CPU), etc., and a driver such as a motor or an actuator, etc. The processing circuitry includes, as hardware resources, a processor, such as a CPU or a micro processing unit (MPU), and a memory, such as a read only memory (ROM) or a random access memory (RAM). The controller 15 can be realized by an application specific integrated circuit (ASIC), a field programmable gate array (FPGA), or another complex programmable logic device (CPLD) or simple programmable logic device (SPLD). The controller 15 controls the X-ray high voltage device 14 and the DAS 18, etc. in accordance with instructions from the console 40. The processor implements the above control by reading and executing a program stored in the memory.

The CPU can execute a computer program including a set of computer-readable instructions that perform the functions described herein, and the program is stored in any of the above-described non-transitory electronic memories and/or a hard disk drive, CD, DVD, FLASH drive or any other known storage media. Further, the computer-readable instructions may be provided as a utility application, background daemon, or component of an operating system, or combination thereof, executing in conjunction with a processor and an operating system known to those skilled in the art. Further, the CPU can be implemented as multiple processors cooperatively working in parallel to perform the instructions.

The controller 15 also has the function of performing operation control of the gantry 10 and the bed 30 in response to an input signal from an input interface 43 to be described later attached to the console 40 or the gantry 10. For example, the controller 15 performs control to rotate the rotation frame 13, control to tilt the gantry 10, or control to operate the bed 30 and the table top 33 in response to an input signal. The control to tilt the gantry 10 is implemented by the controller 15 rotating the rotation frame 13 around an axis parallel to the X-axis direction, based on tilt angle information input through the input interface 43 attached to the gantry 10. The controller 15 may be provided either in the gantry 10 or in the console 40. The controller 15 may be configured by directly integrating a program in the circuitry of the processor, instead of storing a program in the memory. In this case, the processor implements the above-described control by reading and executing the program integrated in the circuitry.

The wedge filter 16 is a filter for adjusting the dose of X-rays emitted from the X-ray tube 11. Specifically, the wedge filter 16 is a filter that allows X-rays emitted from the X-ray tube 11 to pass therethrough, and attenuates the X-rays so that the X-rays emitted from the X-ray tube 11 to the subject P exhibit predetermined distribution. For example, the wedge filter 16 (or bow-tie filter) is a filter obtained by processing aluminum so that it has a predetermined target angle and a predetermined thickness.

The collimator 17 is lead plates or the like for narrowing the application range of X-rays that have passed through the wedge filter 16, and includes a slit formed by combining the lead plates or the like. The collimator 17 may be referred to as an “X-ray diaphragm.”

The DAS 18 generates digital data indicating counts of X-rays detected by the X-ray detector 12 (also referred to as “detection data”) for each of a plurality of energy bands (referred to as “energy bins” or simply as “bins”). The detection data is a set of a channel number and row number of a source X-ray detection element, a view number indicating a collected view (also referred to as a projection angle), and data of the count value identified by the energy bin number. The DAS 18 is implemented by, for example, an application specific integrated circuit (ASIC) on which a circuit element capable of generating detection data is mounted. The detection data is transferred to the console 40.

The bed 30 is a device to place thereon the subject P to be scanned and move the subject P, and includes a base 31, a bed actuator 32, a table top 33, and a support frame 34.

The base 31 is a case that supports the support frame 34 movably in the vertical direction.

The bed actuator 32 is a motor or actuator that moves the table top 33 on which the subject P is placed in the longitudinal direction of the table top 33. The bed actuator 32 moves the table top 33 in accordance with control by the console 40 or control by the controller 15. For example, the bed actuator 32 moves the table top 33 in the direction orthogonal to the subject P so that the body axis of the subject P placed on the table top 33 matches the central axis of the bore of the rotation frame 13. The bed actuator 32 may also move the table top 33 in the body axis direction of the subject P in accordance with X-ray CT imaging performed using the gantry 10. The bed actuator 32 generates power by driving at a rotation speed corresponding to the duty ratio of the drive signal from the controller 15. The bed actuator 32 is implemented by a motor, such as a direct drive motor or a servo motor.

The table top 33 provided on the top surface of the support frame 34 is a plate on which the subject P is placed. The bed actuator 32 may move not only the table top 33 but the support frame 34 in the longitudinal direction of the table top 33.

The console 40 includes a memory 41, a display 42, an input interface 43, and processing circuitry 44. Data communication between the memory 41, the display 42, the input interface 43, and the processing circuitry 44 is performed via a bus. The console 40 is described as being separate from the gantry 10, but the gantry 10 may include the console 40 or part of each constituent element of the console 40.

The memory 41 is a storage device, such as a hard disk drive (HDD), a solid state drive (SSD), or an integrated circuit storage device, etc., which stores various types of information. The memory 41 stores, for example, projection data and reconstructed image data. The memory 41 may be not only the HDD, SSD, or the like, but a driver that writes and reads various types of information in and from, for example, a portable storage medium such as CD, DVD, or a flash memory, or a semiconductor memory such as a random access memory (RAM). The storage area of the memory 41 may be in the X-ray CT apparatus 1, or in an external storage device connected via the network. For example, the memory 41 stores data of a CT image or a display image. The memory 41 also stores a control program according to the present embodiment.

The display 42 displays various types of information. For example, the display 42 outputs a graphical user interface (GUI) or the like for receiving a medical image (CT image) generated by the processing circuitry 44, and various types of operations from the operator. For the display 42, for example, a liquid crystal display (LCD), a cathode ray tube (CRT) display, an organic electro luminescence display (OELD), a plasma display, or any other display can be used as appropriate. The display 42 may be provided in the gantry 10. The display 42 may either be a desktop type or configured by a tablet device capable of wirelessly communicating with the console 40.

The input interface 43 receives various types of input operations from the operator, converts a received input operation into an electrical signal, and outputs the electrical signal to the processing circuitry 44. For example, the input interface 43 receives, from the operator, a collection condition for collecting projection data, a reconstruction condition for reconstructing a CT image, and an image-processing condition for generating a post-processing image from the CT image, etc. For the input interface 43, for example, a mouse, a keyboard, a trackball, a switch, a button, a joystick, a touch pad, or a touch panel display can be used as appropriate. In the present embodiment, the input interface 43 does not necessarily include a physical operation component such as a mouse, a keyboard, a trackball, a switch, a button, a joystick, a touch pad, or a touch panel display. For example, the input interface 43 also includes electrical signal processing circuitry that receives an electrical signal corresponding to an input operation from an external input device provided separately from the apparatus, and outputs the electrical signal to the processing circuitry 44. The input interface 43 may be provided in the gantry 10. The input interface 43 may be configured by a tablet device capable of wirelessly communicating with the console 40.

The processing circuitry 44 controls the overall operation of the X-ray CT apparatus 1 in accordance with the electrical signal of the input operation output from the input interface 43. For example, the processing circuitry 44 includes, as hardware resources, a processor such as a CPU, an MPU, or a graphics processing unit (GPU), and a memory such as a ROM or a RAM. With a processor that executes a program loaded into the memory, the processing circuitry 44 performs a system control function 441, a pre-processing function 442, a reconstruction function 443, and a display control function 444. Each of the functions (the system control function 441, the pre-processing function 442, the reconstruction function 443, and the display control function 444) is not necessarily implemented by a single processing circuit. Processing circuitry can be configured by combining a plurality of independent processors, and the processors can execute respective programs to implement the functions.

The system control function 441 controls each function of the processing circuitry 44 based on an input operation received from the operator via the input interface 43. Specifically, the system control function 441 reads a control program stored in the memory 41, loads it into a memory in the processing circuitry 44, and controls each part of the X-ray CT apparatus 1 in accordance with the loaded control program. For example, the processing circuitry 44 performs each function of the processing circuitry 44 based on an input operation received from the operator via the input interface 43. For example, the system control function 441 obtains a two-dimensional positioning image of the subject P to determine the scan range, imaging condition, etc. The positioning image can also be referred to as a “scanogram” or “scout image.”

The pre-processing function 442 generates data obtained by performing pre-processing on detection data output from the DAS 18, such as logarithmic conversion processing, offset correction processing, processing for sensitivity correction between channels, beam hardening correction, and correction for detector calibrations, detector nonlinearities, polar effects, noise balancing, and material decomposition. Data (detection data) before pre-processing and data after pre-processing can be collectively referred to as “projection data.” The pre-processing function 442 is an example of the pre-processor.

The reconstruction function 443 generates CT image data by performing reconstruction processing using a filtered back projection method, a successive approximation reconstruction method, a stochastic image reconstruction method, or the like, on the projection data generated by the pre-processing function 442. The reconstruction function 443 is an example of the reconstruction processor. Image filtering, smoothing, volume rendering, or image differential processing can be applied to the CT image data if required. The display control function 444 converts CT image data generated by the reconstruction function 443 into tomographic image data of a given cross section, or three-dimensional image data by a publicly-known method, based on the input operation received from the operator via the input interface 43. The generation of three-dimensional image data can be performed directly by the reconstruction function 443. The display control function 444 is an example of the display controller.

In one implementation, the X-ray tube 11 is a single source emitting a broad spectrum of X-ray energies, and the PCD 12 can use a direct X-ray radiation detectors based on semiconductors, such as cadmium telluride (CdTe), cadmium zinc telluride (CZT), silicon (Si), mercuric iodide (HgI2), and gallium arsenide (GaAs). As mentioned above, semiconductor-based direct X-ray detectors generally have much faster time response than indirect detectors, such as scintillator detectors. The fast time response of direct detectors enables them to resolve individual X-ray detection events, although at the high X-ray fluxes typical in clinical X-ray applications, some pileup of detection events may occur. The energy of a detected X-ray is proportional to the signal generated by the direct detector, and the detection events can be organized into energy bins yielding spectrally resolved X-ray data for spectral CT.

Numerous modifications and variations of the embodiments presented herein are possible in light of the above teachings. It is therefore to be understood that within the scope of the claims, the application may be practiced otherwise than as specifically described herein. The inventions are not limited to the examples that have just been described; it is in particular possible to combine features of the illustrated examples with one another in variants that have not been illustrated.

Embodiments of the present disclosure may also be as set forth in the following parentheticals.
(1) A method for determining a model characterizing an output from a photon-counting detector (PCD) used in a computed tomography (CT) system, the PCD having a plurality of pixels, the method comprising: constructing a PCD output model that has a plurality of model parameters including a first model parameter set, where the first model parameter set is dependent on an incident count rate on the PCD, and dependent on a pixel position in the PCD; receiving calibration data acquired by scanning a plurality of combinations of basis materials, under a plurality of incident count rates; and estimating the plurality of model parameters based on the received calibration data.
(2) The method of (1), wherein the first model parameter set includes a first subparameter set that is dependent on the incident count rate, and a second subparameter set that is dependent on the pixel position in the PCD, and the estimating step further comprises: estimating initial values of the plurality of model parameters, such that a total count error between an output derived from the PCD output model and the received calibration data is minimized, determining the first subparameter set, such that a global model-data mismatch over the plurality of pixels is minimized, and determining the second subparameter set, such that pixel-to-pixel variations are minimized.
(3) The method of (1), wherein the first model parameter set is one of: a detector deadtime set of the PCD, a charge sharing matrix set of the PCD, and a threshold energy vector set of the PCD.
(4) The method of (1), wherein the constructing step further comprises: determining, as the constructed PCD output model, a cascaded model having submodels characterizing an attenuation effect of the materials, a charging sharing effect, a pulse pileup effect, and an energy binning operation, respectively.
(5) The method of (1), further comprising: receiving verification data acquired by scanning the plurality of combinations of basis materials, under the plurality of incident count rates; and assessing, based on the received verification data, performance of the PCD output model having the estimated plurality of model parameters.
(6) The method of (1), further comprising: receiving a number of parameters specifying a working condition of the PCD; and generating, based on the received number of parameters, a simulated output of the PCD, using the PCD output model with the estimated plurality of model parameters.
(7) The method of (1), further comprising: receiving scanning data acquired by scanning an imaging object using the CT system; correcting the received scanning data or compensating for an effect of a spectral distortion included in the received scanning data, based on the PCD output model with the estimated plurality of model parameters; and reconstructing, based on the corrected scanning data, an image of the imaging object.
(8) An apparatus for determining a model characterizing an output from a photon-counting detector (PCD) used in a computed tomography (CT) system, the PCD having a plurality of pixels, the apparatus comprising: processing circuitry configured to construct a PCD output model that has a plurality of model parameters including a first model parameter, where the first model parameter set is dependent on an incident count rate on the PCD, and dependent on a pixel position in the PCD; receive calibration data acquired by scanning a plurality of combinations of basis materials, under a plurality of incident count rates; and estimate the plurality of model parameters based on the received calibration data.
(9) The apparatus of (8), wherein the first model parameter set includes a first subparameter set that is dependent on the incident count rate, and a second subparameter set that is dependent on the pixel position in the PCD, and the processing circuitry is configured to: estimate initial values of the plurality of model parameters, such that a total count error between the output derived from the PCD output model and the received calibration data is minimized, determine the first subparameter set, such that a global model-data mismatch over the plurality of pixels, is minimized, and determine the second subparameter set such that pixel-to-pixel variations are minimized.
(10) The apparatus of (8), wherein the first model parameter set is one of: a detector deadtime set of the PCD, a charge sharing matrix set of the PCD, and a threshold energy vector set of the PCD.
(11) The apparatus of (8), wherein the processing circuitry is configured to: determine, as the constructed PCD output model, a cascaded model having submodels characterizing an attenuation effect of the materials, a charging sharing effect, a pulse pileup effect, and an energy binning operation, respectively.
(12) The apparatus of (8), wherein the processing circuitry is configured to: receive verification data acquired by scanning the plurality of combinations of basis materials, under the plurality of incident count rates; and assess, based on the received verification data, performance of the PCD output model having the estimated plurality of model parameters.
(13) The apparatus of (8), wherein the processing circuitry is configured to: receive a number of parameters specifying a working condition of the PCD; and generate, based on the received number of parameters, a simulated output of the PCD, using the PCD output model with the estimated plurality of model parameters.
(14) The apparatus of (8), wherein the processing circuitry is configured to: receive scanning data acquired by scanning an imaging object using the CT system; correct the received scanning data or compensate for an effect of a spectral distortion included in the received scanning data, based on the PCD output model with the estimated plurality of model parameters; and reconstruct, based on the corrected scanning data, an image of the imaging object.
(15) A non-transitory computer-readable medium storing a program that, when executed by processing circuitry, causes the processing circuitry to execute a method for determining a model characterizing an output from a photon-counting detector (PCD) used in a computed tomography (CT) system, the PCD having a plurality of pixels, the method comprising: constructing a PCD output model that has a plurality of model parameters including a first model parameter set, where the first model parameter set is dependent on an incident count rate on the PCD, and dependent on a pixel position in the PCD; receiving calibration data acquired by scanning a plurality of combinations of basis materials, under a plurality of incident count rates; and estimating the plurality of model parameters based on the received calibration data.
(16) The non-transitory computer-readable medium of (15), wherein the first model parameter set includes a first subparameter set that is dependent on the incident count rate, and a second subparameter set that is dependent on the pixel position in the PCD, and the estimating step further comprises: estimating initial values of the plurality of model parameters, such that a total count error between the output derived from the PCD output model and the received calibration data is minimized, determining the first subparameter set, such that a global model-data mismatch over the plurality of pixels, is minimized, and determining the second subparameter set such that pixel-to-pixel variations are minimized.
(17) The non-transitory computer-readable medium of (15), wherein the first model parameter set is one of: a detector deadtime set of the PCD, a charge sharing matrix set of the PCD, and a threshold energy vector set of the PCD.
(18) The non-transitory computer-readable medium of (15), wherein the constructing step further comprises: determining, as the constructed PCD output model, a cascaded model having submodels characterizing an attenuation effect of the materials, a charging sharing effect, a pulse pileup effect, and an energy binning operation, respectively.
(19) The non-transitory computer-readable medium of (15), wherein the method further comprises: receiving a number of parameters specifying a working condition of the PCD; and generating, based on the received number of parameters, a simulated output of the PCD, using the PCD output model with the estimated plurality of model parameters.
(20) The non-transitory computer-readable medium of (15), wherein the method further comprises: receiving scanning data acquired by scanning an imaging object using the CT system; correcting the received scanning data or compensating for an effect of a spectral distortion included in the received scanning data, based on the PCD output model with the estimated plurality of model parameters; and reconstructing, based on the corrected scanning data, an image of the imaging object.

Numerous modifications and variations of the embodiments presented herein are possible in light of the above teachings. It is therefore to be understood that within the scope of the claims, the disclosure may be practiced otherwise than as specifically described herein.

1. A method for determining a model characterizing an output from a photon-counting detector (PCD) used in a computed tomography (CT) system, the PCD having a plurality of pixels, the method comprising:
constructing a PCD output model that has a plurality of model parameters including a first model parameter set, where the first model parameter set is dependent on an incident count rate on the PCD, and dependent on a pixel position in the PCD;
receiving calibration data acquired by scanning a plurality of combinations of basis materials, under a plurality of incident count rates; and
estimating the plurality of model parameters based on the received calibration data.

2. The method of claim 1, wherein the first model parameter set includes a first subparameter set that is dependent on the incident count rate, and a second subparameter set that is dependent on the pixel position in the PCD, and the estimating step further comprises:
estimating initial values of the plurality of model parameters, such that a total count error between an output derived from the PCD output model and the received calibration data is minimized,
determining the first subparameter set, such that a global model-data mismatch over the plurality of pixels is minimized, and
determining the second subparameter set, such that pixel-to-pixel variations are minimized.

3. The method of claim 1, wherein the first model parameter set is one of:
a detector deadtime set of the PCD,
a charge sharing matrix set of the PCD, and
a threshold energy vector set of the PCD.

4. The method of claim 1, wherein the constructing step further comprises:
determining, as the constructed PCD output model, a cascaded model having submodels characterizing an attenuation effect of the materials, a charging sharing effect, a pulse pileup effect, and an energy binning operation, respectively.

5. The method of claim 1, further comprising:
receiving verification data acquired by scanning the plurality of combinations of basis materials, under the plurality of incident count rates; and
assessing, based on the received verification data, performance of the PCD output model having the estimated plurality of model parameters.

6. The method of claim 1, further comprising:
receiving a number of parameters specifying a working condition of the PCD; and
generating, based on the received number of parameters, a simulated output of the PCD, using the PCD output model with the estimated plurality of model parameters.

7. The method of claim 1, further comprising:
receiving scanning data acquired by scanning an imaging object using the CT system;
correcting the received scanning data or compensating for an effect of a spectral distortion included in the received scanning data, based on the PCD output model with the estimated plurality of model parameters; and
reconstructing, based on the corrected scanning data, an image of the imaging object.

8. An apparatus for determining a model characterizing an output from a photon-counting detector (PCD) used in a computed tomography (CT) system, the PCD having a plurality of pixels, the apparatus comprising:
processing circuitry configured to
construct a PCD output model that has a plurality of model parameters including a first model parameter, where the first model parameter set is dependent on an incident count rate on the PCD, and dependent on a pixel position in the PCD;
receive calibration data acquired by scanning a plurality of combinations of basis materials, under a plurality of incident count rates; and
estimate the plurality of model parameters based on the received calibration data.

9. The apparatus of claim 8, wherein the first model parameter set includes a first subparameter set that is dependent on the incident count rate, and a second subparameter set that is dependent on the pixel position in the PCD, and the processing circuitry is configured to:
estimate initial values of the plurality of model parameters, such that a total count error between the output derived from the PCD output model and the received calibration data is minimized,
determine the first subparameter set, such that a global model-data mismatch over the plurality of pixels, is minimized, and
determine the second subparameter set such that pixel-to-pixel variations are minimized.

10. The apparatus of claim 8, wherein the first model parameter set is one of:
a detector deadtime set of the PCD,
a charge sharing matrix set of the PCD, and
a threshold energy vector set of the PCD.

11. The apparatus of claim 8, wherein the processing circuitry is configured to:
determine, as the constructed PCD output model, a cascaded model having submodels characterizing an attenuation effect of the materials, a charging sharing effect, a pulse pileup effect, and an energy binning operation, respectively.

12. The apparatus of claim 8, wherein the processing circuitry is configured to:
receive verification data acquired by scanning the plurality of combinations of basis materials, under the plurality of incident count rates; and
assess, based on the received verification data, performance of the PCD output model having the estimated plurality of model parameters.

13. The apparatus of claim 8, wherein the processing circuitry is configured to:
receive a number of parameters specifying a working condition of the PCD; and
generate, based on the received number of parameters, a simulated output of the PCD, using the PCD output model with the estimated plurality of model parameters.

14. The apparatus of claim 8, wherein the processing circuitry is configured to:
receive scanning data acquired by scanning an imaging object using the CT system;
correct the received scanning data or compensate for an effect of a spectral distortion included in the received scanning data, based on the PCD output model with the estimated plurality of model parameters; and
reconstruct, based on the corrected scanning data, an image of the imaging object.

15. A non-transitory computer-readable medium storing a program that, when executed by processing circuitry, causes the processing circuitry to execute a method for determining a model characterizing an output from a photon-counting detector (PCD) used in a computed tomography (CT) system, the PCD having a plurality of pixels, the method comprising:
constructing a PCD output model that has a plurality of model parameters including a first model parameter set, where the first model parameter set is dependent on an incident count rate on the PCD, and dependent on a pixel position in the PCD;
receiving calibration data acquired by scanning a plurality of combinations of basis materials, under a plurality of incident count rates; and
estimating the plurality of model parameters based on the received calibration data.

16. The non-transitory computer-readable medium of claim 15, wherein the first model parameter set includes a first subparameter set that is dependent on the incident count rate, and a second subparameter set that is dependent on the pixel position in the PCD, and the estimating step further comprises:
estimating initial values of the plurality of model parameters, such that a total count error between the output derived from the PCD output model and the received calibration data is minimized,
determining the first subparameter set, such that a global model-data mismatch over the plurality of pixels, is minimized, and
determining the second subparameter set such that pixel-to-pixel variations are minimized.

17. The non-transitory computer-readable medium of claim 15, wherein the first model parameter set is one of:
a detector deadtime set of the PCD,
a charge sharing matrix set of the PCD, and
a threshold energy vector set of the PCD.

18. The non-transitory computer-readable medium of claim 15, wherein the constructing step further comprises:
determining, as the constructed PCD output model, a cascaded model having submodels characterizing an attenuation effect of the materials, a charging sharing effect, a pulse pileup effect, and an energy binning operation, respectively.

19. The non-transitory computer-readable medium of claim 15, wherein the method further comprises:
receiving a number of parameters specifying a working condition of the PCD; and
generating, based on the received number of parameters, a simulated output of the PCD, using the PCD output model with the estimated plurality of model parameters.

20. The non-transitory computer-readable medium of claim 15, wherein the method further comprises:
receiving scanning data acquired by scanning an imaging object using the CT system;
correcting the received scanning data or compensating for an effect of a spectral distortion included in the received scanning data, based on the PCD output model with the estimated plurality of model parameters; and
reconstructing, based on the corrected scanning data, an image of the imaging object.
